(19) United States
(12) Patent Application Publication
(10) Pub. No.: US 2021/0196408 A1
(43) Pub. Date: Jul. 1, 2021

(54) MANUAL INSTRUMENT RETRACTOR FOR A ROBOTIC SURGERY SYSTEM

(71) Applicant: Titan Medical Inc., Toronto (CA)

(72) Inventors: Daniel Hoffman, Raleigh, NC (US); Tyler Michael, Raleigh, NC (US); Abraham Shipley, Raleigh, NC (US)

(21) Appl. No.: 16/731,600

(22) Filed: Dec. 31, 2019

(51) Int. Cl. A61B 34/30; A61B 17/3201; A61B 17/29; A61B 46/10

(52) U.S. Cl. CPC A61B 34/30; A61B 17/3201; A61B 2017/00477; A61B 46/10; A61B 2017/291; A61B 17/2909

(57) ABSTRACT

An instrument support apparatus for use in a robotic surgery system is disclosed. The apparatus includes a housing, and a mounting interface disposed on the housing, the mounting interface being operably configured to receive and support a proximal end of an elongate shaft of a surgical instrument, the instrument including a tool disposed at a distal end of the elongate shaft. The apparatus also includes an automated drive coupled to the proximal end of the instrument and controlled by the robotic surgery system to cause movement of the proximal end for extending or retracting the instrument to position the tool within a surgical operating site. The apparatus further includes a manual actuator disposed on an accessible location on the housing and configured to be rotated by an operator to cause a manual retraction of the instrument in the event of a failure of the automated drive.

### BACKGROUND

### 1. Field

This disclosure relates generally to robotic surgery systems and more specifically to a manual instrument retractor used in such systems.

### 2. Description of Related Art

In the event of a failure in a robotic surgery system, actuators that cause manipulation of tools for performing operations in a surgical site should generally revert to a safe condition in which no further forces are supplied to actuators and the tool is thus inactivated. Under these conditions the tools may be rendered inactive, but would remain within the surgical site. There remains a need for systems and methods for safely withdrawing the inactivated tools from the surgical site in the event of a failure.

### SUMMARY

An instrument support apparatus for use in a robotic surgery system may include a housing, and a mounting interface disposed on the housing, the mounting interface configured to receive and support a proximal end of an elongate shaft of a surgical instrument, the surgical instrument including a tool disposed at a distal end of the elongate shaft. The apparatus also includes an automated drive coupled to the proximal end of the surgical instrument and controlled by the robotic surgery system to cause movement of the proximal end for extending or retracting the surgical instrument to position the tool within a surgical operating site. The apparatus further includes a manual actuator disposed on an accessible location on the housing and configured to be rotated by an operator to cause a manual retraction of the surgical instrument in the event of a failure of the automated drive.

The accessible location may be an exterior surface of the housing.

The manual actuator may be configured to be rotated by a hand of the operator.

The automated drive may be coupled to the mounting interface and be configured to cause movement of the mounting interface with respect to the housing, the movement of the mounting interface causing movement of the proximal end of the surgical instrument.

The automated drive may include a motor coupled via a leadscrew to the mounting interface, the motor being controlled by the robotic surgery system for extending or retracting the surgical instrument, and the manual actuator may be configured to couple to the leadscrew to cause rotation of the leadscrew when the manual actuator is rotated.

The manual actuator may be coupled to the leadscrew via a releasable coupling that is configured to be engaged with the leadscrew in response to the manual actuator being engaged by the operator.

The releasable coupling may be further configured to inhibit an extending movement of the surgical instrument to prevent the tool from being further extended within the surgical site.

The manual actuator may include a rotatable wheel including an indented recess offset from a rotational axis of the wheel and configured to accommodate an operator's finger for causing rotation of the wheel.

The indented recess may be configured to permit operation of the rotatable wheel through a sterile drape covering the housing.

During a surgical procedure, the housing may be covered by a sterile drape made of a flexible material, and the manual actuator may be configured to be accessible through the sterile drape.

The mounting interface may include a plurality of mounting interfaces, each being configured to receive and support a respective surgical instrument, the automated drive may include a respective plurality of automated drives coupled to proximal ends of respective surgical instruments, and the manual actuator may include a plurality of manual actuators associated with each of the respective surgical instruments.

When the surgical instrument is mounted on the mounting interface, the distal end of the elongate shaft may protrude beyond a front portion of the housing, and the manual actuator may be disposed on a rear surface of the housing.

The tool of the surgical instrument may include an end effector, the end effector being actuable in response to receiving an actuation force via a control link extending between the proximal end and the end effector, and the mounting interface may include a coupler configured to engage the control link at the proximal end, the coupler being driven from within the housing to transmit the actuation force to the control link for actuating the end effector.

The shaft of the surgical instrument may include a dexterous portion actuated by one or more control links to cause a bending movement of the elongate shaft for positioning the tool within the surgical site, and the mounting interface may include a plurality of couplers configured to couple to the respective control links to cause movement of the dexterous portion when the proximal end of the surgical instrument is received in the mounting interface.

The robotic surgery system may be configured to cause the control links to be returned to a state that causes the dexterous portion of the elongate shaft to straighten to facilitate the manual retraction.

Disclosed are methods of operating the robotic surgery system of any of the preceding paragraphs and/or any of the robotic surgery systems described herein.

Other aspects and features will become apparent to those ordinarily skilled in the art upon review of the following description of specific disclosed embodiments in conjunction with the accompanying figures.

### BRIEF DESCRIPTION OF THE DRAWINGS

In drawings which illustrate disclosed embodiments,

FIG. 1 is a rear perspective view of an instrument support apparatus;

FIG. 2 is a rear perspective view of an underside of the instrument support apparatus shown in FIG. 1;

FIG. 3 is a perspective view of an automated drive and a manual actuator; and

FIG. 4 is a perspective view of the instrument support apparatus of FIG. 1 with a sterile drape in place.

### DETAILED DESCRIPTION

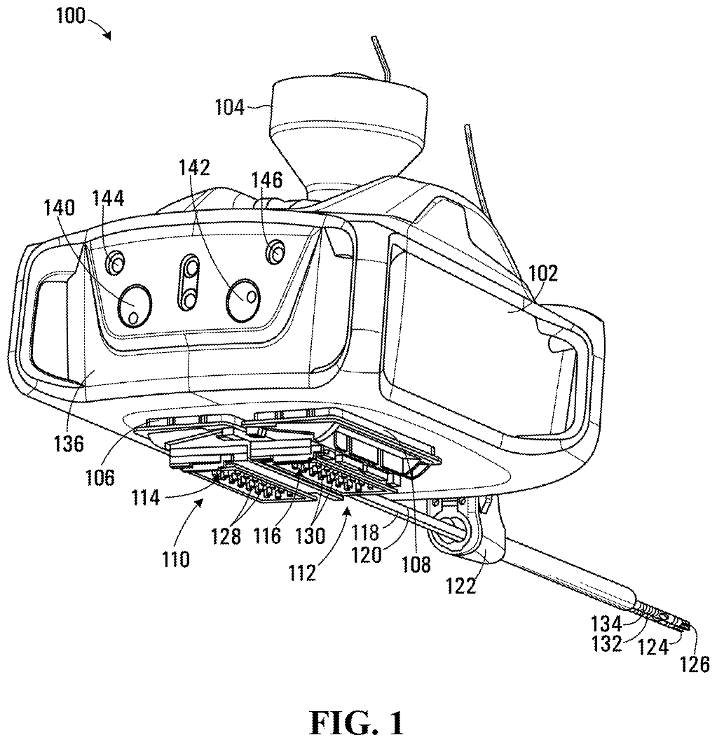

Referring to FIG. 1, an instrument support apparatus according to a first disclosed embodiment is shown generally at 100. The apparatus 100 includes a housing 102 suspended via a column 104, which would generally be coupled to a positioning arm or other support structure of a robotic surgery system (not shown) that facilitates positioning of the instrument support apparatus for performing surgical operations. In the embodiment shown, the apparatus 100 includes a pair of mounting interfaces 106 and 108 for mounting first and second surgical instruments 110 and 112 in a side-by-side arrangement. Each of the first and second surgical instruments 110 and 112 includes a proximal or actuator end 114 and 116, that couple to the respective mounting interfaces 106 and 108. The instruments 110 and 112 each include respective elongate shafts 118 and 120, that extend through a guide 122 mounted to the housing 102. The elongate shafts 118 and 120 each have a tool 124 and 126 disposed at a distal end of each respective shaft. The instruments 110 and 112 may be configured to mount any one of a plurality of different tools, such as a dissection hook, scissor, forceps, etc.

In other embodiments, the apparatus 100 may have a single mounting interface for mounting a single surgical instrument or the apparatus may include more than two mounting interfaces, each being configured to receive and support a respective surgical instrument.

The proximal ends 114 and 116 of the instruments 110 and 112 each include a respective plurality of actuators 128 and 130 configured to interface to a plurality of drivers of the apparatus 100. One or more of the actuators 128 and 130 on each instrument may be coupled via a control link (not shown) extending through the shaft 118 or 120 to the respective tools 124 or 126. The control links are actuable by moving one or more of the actuators 128 and 130 to cause movement of the tools 124 and 126 to perform end effector functions such as grasping for a pair of forceps, or a cutting action for a scissor end effector.

In the embodiment shown, each of the shafts 118, 120 includes a respective dexterous portion 132 and 134 actuated by one or more additional control links coupled to the dexterous portions. The control links are each coupled at the proximal ends 114 and 116 of the instruments 110 and 112 to respective actuators 128 and 130. The actuators 128 and 130 cause movement of the control links that in turn cause bending movements of the dexterous portions 132 and 134 for positioning the tools 124 and 126 within a surgical site. The apparatus 100 forms part of a robotic surgery system, which controls the first and second surgical instruments 110 and 112 via the instrument support apparatus.

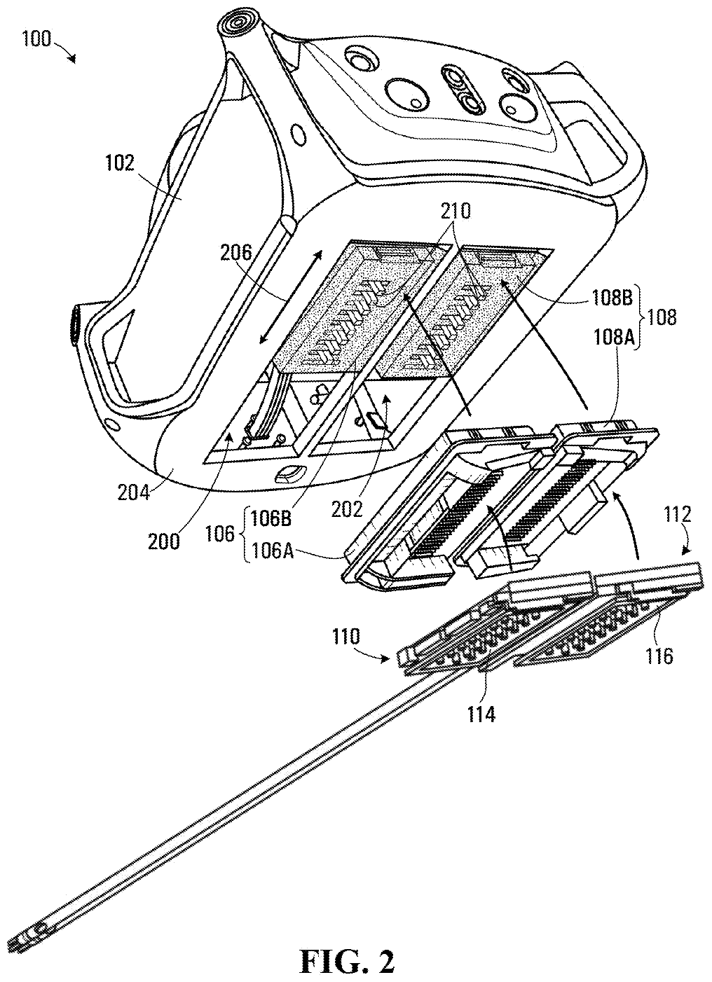

The apparatus 100 is shown in FIG. 2 with the instruments 110 and 112 removed. To maintain sterility of the surgical instruments 110 and 112, in the embodiment shown, the mounting interfaces 106 and 108 each include two separable portions 106A, 106B, and 108A, 108B. The portions 106A and 108A are provided as sterile interfaces that are received on interface portions 106B and 108B, which may not be sterile (for example, due to particular difficulty of sterilizing the apparatus 100). Each of the sterile adapters 106A and 108A may be removably attached to the interface portions 106B and 108B. The instruments 110 and 112 may thus be mounted to the sterile adapters 106A and 108A, which have the same or substantially similar length and width as the instrument interface portions 106B and 108B. The sterile adapters 106A and 108A are in turn mounted to the interface portions 106B and 108B. The sterile adaptors 106A and 108A may be packaged as sterile components and may be disposed of after performing a surgery or may be sterilized and re-used.

The interface portions 106B and 108B of the apparatus 100 are disposed within openings 200 and 202 in a bottom surface 204 of the housing 102. The interface portions 106B and 108B are moveable back and forth within the openings 200 and 202 in a direction indicated by the arrow 206. Movement of each of the interface portions 106B and 108B causes corresponding movements of the instruments 110 and 112 for extending or retracting the instruments 110 and 112. The interface portions 106B and 108B are each coupled to a corresponding automated drive within the housing 102 configured to cause movements of the mounting interfaces with respect to the housing. The automated drive is thus coupled via the interface portions 106B and 108B and the sterile adaptors 106A and 108A to the proximal ends 114 and 116 of the instruments 110 and 112 and controlled by the robotic surgery system to cause movement of the proximal ends for extending or retracting the instrument to position the tools 124 and 126 within a surgical operating site. The robotic surgery system can include at least one controller configured to cause movement of the proximal ends for extending or retracting the instrument as described herein. The at least one controller can be a processor, ASIC, FPGA, or dedicated hardware and may include logic circuitry.

The interface portions 106B and 108B include protrusions 210 that are able to interface with the actuators 128 and 130 of the instruments 110 and 112 via the sterile adaptors 108A and 108B. The protrusions 210 are coupled to and driven by additional drive components within the housing 102 to provide forces for moving the dexterous portions 132 and 134 and for actuating the end effector tools 124 and 126.

Referring back to FIG. 1, the apparatus 100 includes a pair of manual actuators 140 and 142. The manual actuator 140 is associated with the mounting interface 106 and the manual actuator 142 is associated with the mounting interface 108. The manual actuators 140 and 142 are disposed on an accessible location on the housing 102 and are each configured to be rotated by hand to cause a manual retraction of the respective instruments 110 and 112 in the event of a failure of the automated drives within the housing 102. In the embodiment shown, the manual actuators 140 and 142 are disposed on a rear surface 136 of the housing 102. As is shown in FIG. 1, the manual actuators 140 and 142 can be disposed at least partially on the exterior surface of the housing 102.

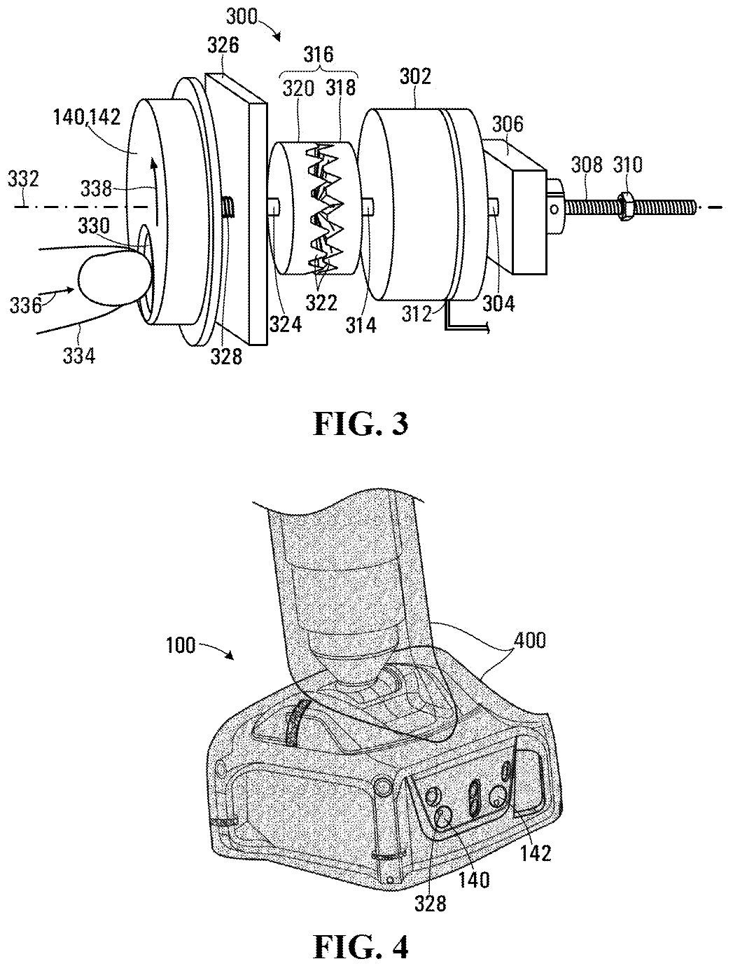

Referring to FIG. 3, an automated drive and manual actuator associated with either one of the interface portions 106B or 108B is shown generally at 300. The automated drive 300 includes a motor 302, having a shaft 304 coupled via a gearbox 306 to a leadscrew 308. A leadscrew nut 310 is received on the leadscrew 308 and would be coupled to the interface portion 106B or 108B (not shown in FIG. 3). The motor 302 includes a control input 312 for receiving a control signal from the robotic surgery system (for example, from the at least one controller) to cause the leadscrew 308 to rotate. The rotation may be in either an anti-clockwise direction, for extending the leadscrew nut 310 and thus the surgical instrument 110 or 112, or a clockwise direction for retracting the surgical instrument. The gearbox 306 is provided to reduce the rotational speed of the leadscrew 308 for a selected shaft speed of the motor 302 and also to increase the torque delivered to the leadscrew.

Referring back to FIG. 1, in the embodiment shown, the rear surface 136 of the housing 102 includes buttons 144 and 146 that may be pressed by an operator on completion of an operation with a surgical instrument to cause the mounting interfaces 106 and 108 to automatically retract from the surgical site.

Referring again to FIG. 3, the shaft 304 of the motor 302 has a portion 314 that extends rearwardly of the automated drive 300 and is connected to a releasable coupling 316 having a first portion 318 and a second portion 320 each having corresponding teeth 322. The manual actuator 140 or 142 is coupled to the second portion 320 of the coupling 316 via a shaft 324 that passes through a bulkhead plate 326. A spring 328 urges the manual actuator 140 or 142 away from the bulkhead plate 326 such that the corresponding teeth 322 of the first portion 318 and the second portion 320 remain disengaged during normal operation of the robotic surgery system.

In the event of a failure, the robotic surgery system (for example, the at least one controller) can revert to a ‘Safe Torque Off’ (STO) state in which no torque-generating energy is supplied to the motor 302 to prevent unintentional movement. In one embodiment, a STO signal may be asserted by the robotic surgery system to place the system in a safe state. For example, if an operator were to activate a “Stop” button, the STO signal would be asserted and control signals at the input 312 of the motor 302 would permit the motor to move freely. The control signals can be asserted by the at least one controller. In the embodiment shown, the manual actuator 140 or 142 is configured in the shape of a wheel having an indented recess 330 offset from a rotational axis 332 of the wheel. The indented recess 330 is configured to accommodate an operator's finger 334 for causing rotation of the wheel. When it is necessary to manually retract one or both of the instruments 110 and 112, the operator places their finger 334 in the indented recess 330 and urges the manual actuator 140 or 142 in the direction indicated by the arrow 336 to cause the corresponding teeth 322 of the first portion 318 and the second portion 320 of the coupling 316 to engage.

The manual actuator 140 or 142 may then be rotated by hand in a clockwise direction to retract the instrument from the surgical site. The gearbox 306 reduces the torque required for retraction of the instrument. The motor 302, being un-energized, permits free rotation of the shaft 304 and leadscrew 308. The manual actuator 140, 142 is thus only engaged in response to the manual actuator being engaged by the operator's hand.

When the STO signal is asserted, the dexterous portions 132 and 134 of the instruments 110 and 112 may not be disposed in a straightened condition such as shown in FIG. 1 but may be bent off axis to position the respective tools 124 or 126. The asserted STO signal may also signal the actuators 128 and 130 of the respective instruments 110 and 112 to return the dexterous portions 132 and 134 to a straightened condition to facilitate retraction of the shafts 118 and 120 through the guide 122 when the manual actuators 140 and 142 are engaged and rotated to retract the instruments.

In the embodiment shown, the manual actuator 140, 142 has an arrow 338 marked on the wheel of the actuator to indicate the correct anti-clockwise direction for retracting the surgical instrument 110 or 112. In other embodiments, the coupling may be configured to inhibit the manual actuator 140 or 142 from causing an extending movement of the instrument 110 or 112, thus preventing the tool from being further extended into the surgical site. As an example, a ratchet mechanism that prevents anti-clockwise rotation of the shaft 324 may be incorporated at the bulkhead plate 326.

In embodiments having a single mounting interface or more than two mounting interfaces, the apparatus 100 may include a respective plurality of automated drives coupled to proximal ends of respective instruments, each having an associated manual actuator.

Referring to FIG. 4, in some embodiments the apparatus 100 may be covered in a sterile drape 400. The sterile drape 400 provides a sterile barrier between the housing 102 of the apparatus 100 and the patient. The housing 102 may be practically difficult to sterilize, since the apparatus 100 may not be able to withstand cleaning using sterilizing liquids applied to the surfaces of the housing 102. One advantage of the manual actuators 140 and 142 is that the indented recess 330 may permit operation of the wheel of the actuator through a sterile drape 400 covering the housing 102. For example, the sterile drape 400 may be made of a flexible material, and the manual actuator 140, 142 configured to be accessible and operable through the sterile drape. The indented recess 330 of the manual actuators 140 and 142 may thus be engaged and operated through the sterile drape 400, removing the need to provide an opening therethrough, which may compromise the sterile barrier.

The above described manual actuator embodiments provide for safe retraction of surgical instruments in the event of a failure of the robotic surgery system. The manual actuator may remain disengaged until needed and may also be operated without removing or breeching the sterile drape that maintains a sterile barrier between non-sterilized portions of the system and the surgical site.

Systems and methods disclosed herein can be used with one or more features of tool positioners described in U.S. Pat. No. 10,278,683, tool actuators and manipulators described in U.S. Pat. No. 9,629,688, or sterile barriers described in U.S. patent application Ser. No. 16/453,910, filed on Jun. 26, 2019, each of which is incorporated by reference in its entirety.

While specific embodiments have been described and illustrated, such embodiments should be considered illustrative only and not as limiting the disclosed embodiments as construed in accordance with the accompanying claims.

What is claimed is:

1. An instrument support apparatus for use in a robotic surgery system, the apparatus comprising:
a housing;
a mounting interface disposed on the housing, the mounting interface configured to receive and support a proximal end of an elongate shaft of a surgical instrument, the surgical instrument including a tool disposed at a distal end of the elongate shaft;
an automated drive coupled to the proximal end of the surgical instrument and controlled by the robotic surgery system to cause movement of the proximal end for extending or retracting the surgical instrument to position the tool within a surgical operating site; and
a manual actuator disposed on an accessible location on the housing and configured to be rotated by an operator to cause a manual retraction of the surgical instrument in the event of a failure of the automated drive.

2. The apparatus of claim 1 wherein the automated drive is coupled to the mounting interface and is configured to cause movement of the mounting interface with respect to the housing, the movement of the mounting interface causing movement of the proximal end of the surgical instrument.

3. The apparatus of claim 2 wherein the automated drive comprises a motor coupled via a leadscrew to the mounting interface, the motor being controlled by the robotic surgery system for extending or retracting the surgical instrument, and wherein the manual actuator is configured to couple to the leadscrew to cause rotation of the leadscrew when the manual actuator is rotated.

4. The apparatus of claim 3 wherein the manual actuator is coupled to the leadscrew via a releasable coupling configured to be engaged with the leadscrew in response to the manual actuator being engaged by the operator.

5. The apparatus of claim 4 wherein the releasable coupling is further configured to inhibit an extending movement of the surgical instrument to prevent the tool from being further extended within the surgical site.

6. The apparatus of claim 1 wherein the manual actuator comprises a rotatable wheel including an indented recess offset from a rotational axis of the wheel and configured to accommodate an operator's finger for causing rotation of the wheel.

7. The apparatus of claim 6, wherein the indented recess is configured to permit operation of the rotatable wheel through a sterile drape covering the housing.

8. The apparatus of claim 1 wherein during a surgical procedure, the housing is covered by a sterile drape made of a flexible material, and wherein the manual actuator is configured to be accessible through the sterile drape.

9. The apparatus of claim 1 wherein:
the mounting interface comprises a plurality of mounting interfaces, each being configured to receive and support a respective surgical instrument;
the automated drive comprises a respective plurality of automated drives coupled to proximal ends of respective surgical instruments; and
the manual actuator comprises a plurality of manual actuators associated with each of the respective surgical instruments.

10. The apparatus of claim 1 wherein, when the surgical instrument is mounted on the mounting interface, the distal end of the elongate shaft protrudes beyond a front portion of the housing, and wherein the manual actuator is disposed on a rear surface of the housing.

11. The apparatus of claim 1 wherein the tool of the surgical instrument comprises an end effector, the end effector being actuable in response to receiving an actuation force via a control link extending between the proximal end and the end effector, and wherein the mounting interface comprises a coupler configured to engage the control link at the proximal end, the coupler being driven from within the housing to transmit the actuation force to the control link for actuating the end effector.

12. The apparatus of claim 11 wherein the shaft of the surgical instrument comprises a dexterous portion actuated by one or more control links to cause a bending movement of the elongate shaft for positioning the tool within the surgical site, and wherein the mounting interface comprises a plurality of couplers configured to couple to the respective control links to cause movement of the dexterous portion when the proximal end of the surgical instrument is received in the mounting interface.

13. The apparatus of claim 12 wherein the robotic surgery system is configured to cause the control links to be returned to a state that causes the dexterous portion of the elongate shaft to straighten to facilitate the manual retraction.

14. The apparatus of claim 1 wherein the accessible location comprises an exterior surface of the housing.

15. The apparatus of claim 1 wherein the manual actuator is configured to be rotated by a hand of the operator.
